(19) United States
(12) Patent Application Publication
(10) Pub. No.: US 2022/0370839 A1
(43) Pub. Date: Nov. 24, 2022

(54) Filling Element for Filling up a Space Between at Least One Compressed Gas Bottle of a Breathing Apparatus and a Carrier Plate

(71) Applicant: MSA Europe GmbH, Jona (CH)

(72) Inventors: Eric Holtkamp, Berlin (DE); Wolfgang Weber, Berlin (DE)

(21) Appl. No.: 17/640,699

(22) PCT Filed: Sep. 1, 2020
(86) PCT No.: PCT/EP2020/074276

§ 371 Date: Mar. 4, 2022

(30) Foreign Application Priority Data
Sep. 5, 2019 (DE) 10 2019 123 813.7

(51) Int. Cl. A62B 25/00; A62B 9/00; A62B 9/04

(52) U.S. Cl. CPC A62B 25/00; A62B 9/006; A62B 9/04

(57) ABSTRACT

A carrier for carrying at least one compressed gas bottle of a breathing apparatus includes a carrier plate configured for removably receiving the at least one compressed gas bottle, and at least one filling element positioned in a space between the carrier plate and the at least one compressed air bottle. The at least one filling element includes a first positive connection element having a surface for engaging the at least one compressed gas bottle, and a second positive connection element connected to the first positive connection element and the carrier plate.

### CROSS-REFERENCE TO RELATED APPLICATION

This application claims priority to German Patent Application No. 10 2019 123 813.7, filed on Sep. 5, 2019, the disclosure of which is incorporated by reference herein in its entirety.

### BACKGROUND

### Technical Field

This disclosure relates to a filling element for filling up a space between at least one compressed gas bottle of a breathing apparatus and a carrier plate.

### Technical Considerations

Breathing apparatuses that are worn, for example, by firefighters when fighting fires, generally include one or two compressed gas bottles in which breathing gas can be transported under pressure. The compressed gas bottles typically have a circular cylindrical cross-section in their middle part. At one end, in service mostly at the lower end, there is arranged a valve to which breathing gas hoses for the wearer can be connected. At the opposite end, in service mostly at the upper end, the compressed gas bottle generally has a hemispherical termination.

To ensure that the at least one compressed gas bottle is transportable for a person, the compressed gas bottles of substantially cylindrical shape are fixed on a carrier plate, e.g. by means of straps. The carrier plate typically is of substantially flat design, optionally with a certain contouring for the back of the person on which the carrier plate is worn.

Due to the geometries used—cylindrical compressed gas bottle and flat carrier plate—a space is formed between the compressed gas bottle and the carrier plate, in which items (e.g. cables, wires etc.) can get caught in service. This is dangerous for the person on duty, as in service a jammed or tangled item can hinder movement and possibly be difficult to remove from the breathing apparatus, thereby reducing the user's efficiency.

### SUMMARY

Therefore, the present disclosure provides improved devices that facilitate safer working environments.

In some non-limiting embodiments or aspects, a carrier for carrying at least one compressed gas bottle of a breathing apparatus may include a carrier plate configured for removably receiving the at least one compressed gas bottle; and at least one filling element positioned in a space between the carrier plate and the at least one compressed air bottle. The at least one filling element may include a first positive connection element having a surface for engaging the at least one compressed gas bottle, and a second positive connection element connected to the first positive connection element and the carrier plate.

In some non-limiting embodiments or aspects, the at least one filling element may have a length extending in a longitudinal direction along at least a quarter of a length of the carrier plate.

In some non-limiting embodiments or aspects, the at least one filling element may have a wedge shape with a larger depth on an upper side than a lower side.

In some non-limiting embodiments or aspects, the surface of the first positive connection element may be at least partially concave.

In some non-limiting embodiments or aspects, the first positive connection element may have a sealing lip configured for conforming to an outer surface of the at least one compressed gas bottle.

In some non-limiting embodiments or aspects, wherein the first positive connection element may be made from an elastomer material that conforms to the at least one compressed gas bottle.

In some non-limiting embodiments or aspects, the second positive connection element may be latched into a groove of the carrier plate.

In some non-limiting embodiments or aspects, the at least one filling element further may include at least one functional unit.

In some non-limiting embodiments or aspects, the at least one functional unit may be received within a cavity of the at least one filling element.

In some non-limiting embodiments or aspects, the at least one functional unit may be at least one of: a gas sensor, an indicating device configured for indicating an operating condition, a lighting device, an acoustic warning element, a battery device, a radio device, and any combination thereof.

In some non-limiting embodiments or aspects, the indicating device may be at least one of a battery indicator and a level indicator for the at least one compressed gas bottle.

In some non-limiting embodiments or aspects, the lighting device may be configured for signaling a state of the breathing apparatus or as an emergency lighting.

In some non-limiting embodiments or aspects, the at least one filling element further may include at least one receiving element configured for receiving a breathing hose.

In some non-limiting embodiments or aspects, the at least one receiving element may be a depression.

In some non-limiting embodiments or aspects, the at least one filling element further may include a neck guard configured for cooperating with a helmet.

In some non-limiting embodiments or aspects, a breathing apparatus may include at least one compressed gas bottle; a carrier plate configured for removably receiving the at least one compressed gas bottle; and at least one filling element positioned in a space between the carrier plate and the at least one compressed air bottle. The at least one filling element may include a first positive connection element having a surface for engaging the at least one compressed gas bottle, and a second positive connection element at least partially received within the first positive connection element, the second positive connection element being connected to the carrier plate.

In some non-limiting embodiments or aspects, at least one filling element may have a length extending in a longitudinal direction along at least a quarter of a length of the carrier plate, and the at least one filling element may have a wedge shape with a larger depth on an upper side than a lower side.

In some non-limiting embodiments or aspects, the surface of the first positive connection element may be at least partially concave and may have a sealing lip configured for conforming to an outer surface of the at least one compressed gas bottle.

In some non-limiting embodiments or aspects, the at least one filling element further may include at least one functional unit received within a cavity of the at least one filling element, and wherein the at least one functional unit may be at least one of: a gas sensor, an indicating device configured for indicating an operating condition, a lighting device, an acoustic warning element, a battery device, a radio device, and any combination thereof.

In some non-limiting embodiments or aspects, the at least one filling element further may include at least one receiving element configured for receiving a breathing hose.

In some non-limiting embodiments or aspects, a filling element may serve for filling up a space between at least one compressed gas bottle of a breathing apparatus and a carrier plate for the at least one compressed gas bottle, wherein at least one positive connection element serves for connection with the at least one compressed gas bottle.

In some non-limiting embodiments or aspects, the space can be of quite complex shape, as the carrier plate and the at least one compressed gas bottle have rounded and curved parts. The filling element, which also can have several parts, at least partly fills this space so that no other items can get into the space. To this end, the at least one filling element may include a first positive connection element which creates a connection with the at least one compressed gas bottle. This connection need not be load-bearing. It is sufficient when the filling element and the at least one compressed gas bottle are in contact such that an external item cannot penetrate into the space between carrier plate and compressed gas bottle or can only do so with great difficulty.

In some non-limiting embodiments or aspects, the at least one positive connection element may include a sealing lip and/or a foam element for conforming to the at least one compressed gas bottle. When the at least one compressed gas bottle is attached to the carrier plate, the sealing lip can serve to achieve filling of the space between the carrier plate and the at least one compressed gas bottle without leaving a gap.

In some non-limiting embodiments or aspects, it is also possible that the filling element includes at least one second positive connection element for connecting the filling element with the carrier plate. The filling element thereby can be latched into a groove of the carrier plate.

In some non-limiting embodiments or aspects, the filling element may have an extension along the longitudinal axis of the carrier plate, which extends along more than a quarter of the extension of the carrier plate, in particular along half of the longitudinal extension of the carrier plate, quite particularly up to a port of the at least one compressed gas bottle at the lower end of the carrier plate. Depending on the intended use, differently large parts of the space between the carrier plate and the at least one compressed gas bottle can be filled thereby.

Alternatively or in addition, it is also possible that the filling element has an extension along the longitudinal axis of the carrier plate up to the upper end of the carrier plate. In this way, for example the critical area in the neck is particularly filled.

In some non-limiting embodiments or aspects, a simple geometry of one filling element may be of substantially cuboid-shaped or wedge-shaped design, wherein the surface facing the at least one compressed gas bottle may be at least partly concave so that the filling element can be adapted to a compressed gas bottle of substantially circular cylindrical shape.

In some non-limiting embodiments or aspects, the filling element may also include at least one receiving element for a hose line. Such hose lines, for example, can be pressurized breathing air hose lines.

In some non-limiting embodiments or aspects, the filling element may include at least one gas sensor. The space between the carrier plate and the compressed gas bottle may be well-suited for accommodating a gas sensor, as the gas sensor is located close to the body of the person. When the gas sensor is arranged in the upper region of the carrier plate, the gas concentration may be measured in the region of the head, which is particularly safety-relevant.

In some non-limiting embodiments or aspects, there can also be provided an indicating device for an operating condition, in particular a battery indicator and a level indicator for the at least one compressed gas bottle. Moreover, in some non-limiting embodiments or aspects of a filling element, an LED lighting device can be arranged, in particular for signaling a state of the breathing apparatus or as an emergency lighting. In service, this lighting device would not be properly visible for the wearing person, but very well for colleagues who can recognize, e.g., the sinking gas pressure.

It would also be possible when at least one acoustic warning element, in particular a loudspeaker, is provided in some non-limiting embodiments or aspects.

In some non-limiting embodiments or aspects of the filling element, the same includes at least one battery device, in particular a battery device integrated in the interior. As batteries are comparatively heavy, an arrangement close to the carrier plate is expedient.

In some non-limiting embodiments or aspects, the filling element also can include at least one radio device, in particular a locating means, a router and/or a means for producing an ad-hoc network. Here as well, the filling element may provide protection for the integrated devices.

Furthermore, in some non-limiting embodiments or aspects, a filling element can include a neck guard for a wearer of the breathing apparatus, wherein the neck guard in particular can cooperate with a helmet of a wearer of the breathing apparatus.

In some non-limiting embodiments or aspects, the filling element may be made of a plastic material, in particular an elastomer or polymer.

Further non-limiting embodiments or aspects are set forth in the following numbered clauses:

Clause 1: A carrier for carrying at least one compressed gas bottle of a breathing apparatus, the carrier comprising: a carrier plate configured for removably receiving the at least one compressed gas bottle; and at least one filling element positioned in a space between the carrier plate and the at least one compressed air bottle, wherein the at least one filling element comprises: a first positive connection element having a surface for engaging the at least one compressed gas bottle, and a second positive connection element connected to the first positive connection element and the carrier plate.

Clause 2: The carrier of clause 1, wherein at least one filling element has a length extending in a longitudinal direction along at least a quarter of a length of the carrier plate.

Clause 3: The carrier of clause 1 or 2, wherein the at least one filling element has a wedge shape with a larger depth on an upper side than a lower side.

Clause 4: The carrier of any of clauses 1-3, wherein the surface of the first positive connection element is at least partially concave.

Clause 5: The carrier of any of clauses 1-4, wherein the first positive connection element has a sealing lip configured for conforming to an outer surface of the at least one compressed gas bottle.

Clause 6: The carrier of any of clauses 1-5, wherein the first positive connection element is made from an elastomer material that conforms to the at least one compressed gas bottle.

Clause 7: The carrier of any of clauses 1-6, wherein the second positive connection element is latched into a groove of the carrier plate.

Clause 8: The carrier of any of clauses 1-7, wherein the at least one filling element further comprises at least one functional unit.

Clause 9: The carrier of any of clauses 1-8, wherein the at least one functional unit is received within a cavity of the at least one filling element.

Clause 10: The carrier of any of clauses 1-9, wherein the at least one functional unit is at least one of: a gas sensor, an indicating device configured for indicating an operating condition, a lighting device, an acoustic warning element, a battery device, a radio device, and any combination thereof

Clause 11: The carrier of clauses 1-10, wherein the indicating device is at least one of a battery indicator and a level indicator for the at least one compressed gas bottle.

Clause 12: The carrier of clauses 1-11, wherein the lighting device is configured for signaling a state of the breathing apparatus or as an emergency lighting.

Clause 13: The carrier of any of clauses 1-12, wherein the at least one filling element further comprises at least one receiving element configured for receiving a breathing hose.

Clause 14: The carrier of any of clauses 1-13, wherein the at least one receiving element is a depression.

Clause 15: The carrier of any of clauses 1-14, wherein the at least one filling element further comprises a neck guard configured for cooperating with a helmet.

Clause 16: A breathing apparatus comprising: at least one compressed gas bottle; a carrier plate configured for removably receiving the at least one compressed gas bottle; and at least one filling element positioned in a space between the carrier plate and the at least one compressed air bottle, wherein the at least one filling element comprises: a first positive connection element having a surface for engaging the at least one compressed gas bottle, and a second positive connection element at least partially received within the first positive connection element, the second positive connection element being connected to the carrier plate.

Clause 17: The breathing apparatus of clause 16, wherein at least one filling element has a length extending in a longitudinal direction along at least a quarter of a length of the carrier plate, and wherein the at least one filling element has a wedge shape with a larger depth on an upper side than a lower side.

Clause 18: The breathing apparatus of clause 16 or 17, wherein the surface of the first positive connection element is at least partially concave and has a sealing lip configured for conforming to an outer surface of the at least one compressed gas bottle.

Clause 19: The breathing apparatus of any of clauses 16-18, wherein the at least one filling element further comprises at least one functional unit received within a cavity of the at least one filling element, and wherein the at least one functional unit is at least one of: a gas sensor, an indicating device configured for indicating an operating condition, a lighting device, an acoustic warning element, a battery device, a radio device, and any combination thereof.

Clause 20: The breathing apparatus of any of clauses 16-19, wherein the at least one filling element further comprises at least one receiving element configured for receiving a breathing hose.

Clause 21: A filling element (1) for filling up a space (20) between at least one compressed gas bottle (2) of a breathing apparatus and a carrier plate (3) for the at least one compressed gas bottle (2), characterized by at least one first positive connection element (4) for connection with the at least one compressed gas bottle (2).

Clause 22: The filling element (1) according to clause 21, characterized in that the at least one positive connection element (4) includes a sealing lip (5) and/or a foam element for conforming to the at least one compressed gas bottle (2).

Clause 23: The filling element (1) according to clauses 21 or 22, characterized by a second positive connection element (6) for connection with the carrier plate (3).

Clause 24: The filling element (1) according to at least one of clauses 21-23, characterized by an extension along the longitudinal axis of the carrier plate (3), wherein the filling element (1) extends along more than a quarter of the extension of the carrier plate (3), in particular along half of the longitudinal extension of the carrier plate (3), quite particularly up to a port (7) of the at least one compressed gas bottle (2) at the lower end of the carrier plate (3).

Clause 25: The filling element (1) according to at least one of clauses 21-24, characterized by an extension along the longitudinal axis of the carrier plate (3) up to the upper end of the carrier plate (3).

Clause 26: The filling element (1) according to at least one of clauses 21-25, characterized in that it is of substantially wedge-shaped or cuboid-shaped design, wherein the surface facing the at least one compressed gas bottle (2) is at least partly concave so that the filling element (1) is adaptable to a compressed gas bottle (2) of substantially circular cylindrical shape.

Clause 27: The filling element (1) according to at least one of clauses 21-26, characterized by at least one receiving element (8a) for a hose line.

Clause 28: The filling element (1) according to at least one of clauses 21-27, characterized by at least one gas sensor (8).

Clause 29: The filling element (1) according to at least one of clauses 21-28, characterized by at least one indicating device (9) for an operating condition, in particular a battery indicator and a level indicator for the at least one compressed gas bottle (2) and/or a lighting device.

Clause 30: The filling element (1) according to at least one of clauses 21-29, characterized by at least one acoustic warning element (10), in particular a loudspeaker.

Clause 31: The filling element (1) according to at least one of clauses 21-30, characterized by at least one battery device (11), in particular a battery device (11) integrated in the interior.

Clause 32: The filling element (1) according to at least one of clauses 21-31, characterized by at least one radio device (12), in particular a locating means, a router and/or a means for producing an ad-hoc network.

Clause 33: The filling element (1) according to at least one of clauses 21-32, characterized by a neck guard (13) for a wearer of the breathing apparatus, wherein the neck guard (13) in particular is coupled with a helmet of a wearer of the breathing apparatus.

Clause 34: The filling element (1) according to at least one of clauses 21-33, characterized in that it is made of a plastic material, in particular an elastomer.

These and other features and characteristics of the filling element described herein, as well as the methods of manufacture of such masks, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only.

### BRIEF DESCRIPTION OF THE DRAWINGS

By means of exemplary and non-limiting embodiments or aspects, the monitoring device and the monitoring system will be described with reference to the accompanying Figures, in which:

FIG. 1 shows a perspective view of a part of a breathing apparatus with a compressed gas bottle and a filling element in accordance with some non-limiting embodiments or aspects;

FIG. 2 shows a detailed view of the representation in FIG. 1;

FIG. 3A shows a perspective rear view of a filling element in accordance with some non-limiting embodiments or aspects with a connecting element connected to the carrier plate;

FIG. 3B shows a perspective front view of the filling element shown in FIG. 3A;

FIG. 3C shows another perspective front view of the filling element shown in FIGS. 3A and 3B;

FIG. 3D shows a side view of the filling element shown in FIGS. 3A-3C;

FIG. 4 shows a perspective view of a filling element in accordance with some non-limiting embodiments or aspects;

FIG. 5 shows a side view of a breathing apparatus with a filling element in accordance with some non-limiting embodiments or aspects;

FIG. 5A shows a variation of the filing element shown in FIG. 5, wherein the filling element extends along an entire length of the compressed gas bottle;

FIG. 5B shows a sectional view taken along line A-A in FIG. 5A;

FIG. 6 shows a schematic view of a multifunctional filling element in accordance with some non-limiting embodiments or aspects; and

FIG. 7 shows a schematic side view of a filling element with a neck guard.

### DETAILED DESCRIPTION

As used herein, the singular form of “a”, “an”, and “the” include plural referents unless the context clearly dictates otherwise.

Spatial or directional terms, such as “left”, “right”, “inner”, “outer”, “above”, “below”, and the like, relate to the invention as shown in the drawing figures and are not to be considered as limiting as the invention can assume various alternative orientations.

All numbers used in the specification and claims are to be understood as being modified in all instances by the term “about”. By “about” is meant within plus or minus twenty-five percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less. The ranges and/or ratios disclosed herein represent the average values over the specified range and/or ratio.

The terms “first”, “second”, and the like are not intended to refer to any particular order or chronology, but refer to different conditions, properties, or elements.

All documents referred to herein are “incorporated by reference” in their entirety.

The term “at least” is synonymous with “greater than or equal to”.

As used herein, “at least one of” is synonymous with “one or more of”. For example, the phrase “at least one of A, B, or C” means any one of A, B, or C, or any combination of any two or more of A, B, or C. For example, “at least one of A, B, and C” includes A alone; or B alone; or C alone; or A and B; or A and C; or B and C; or all of A, B, and C.

The term “includes” is synonymous with “comprises”.

As used herein, the terms “parallel” or “substantially parallel” mean a relative angle as between two objects (if extended to theoretical intersection), such as elongated objects and including reference lines, that is from 0° to 5°, or from 0° to 3°, or from 0° to 2°, or from 0° to 1°, or from 0° to 0.5°, or from 0° to 0.25°, or from 0° to 0.1°, inclusive of the recited values.

As used herein, the terms “perpendicular” or “substantially perpendicular” mean a relative angle as between two objects at their real or theoretical intersection is from 85° to 90°, or from 87° to 90°, or from 88° to 90°, or from 89° to 90°, or from 89.5° to 90°, or from 89.75° to 90°, or from 89.9° to 90°, inclusive of the recited values.

The discussion of various embodiments or aspects may describe certain features as being “particularly” or “preferably” within certain limitations (e.g., “preferably”, “more preferably”, or “even more preferably”, within certain limitations). It is to be understood that the disclosure is not limited to these particular or preferred limitations but encompasses the entire scope of the various embodiments and aspects described herein.

The disclosure comprises, consists of, or consists essentially of, the following embodiments or aspects, in any combination. Various embodiments or aspects of the disclosure are illustrated in separate drawing figures. However, it is to be understood that this is simply for ease of illustration and discussion. In the practice of the disclosure, one or more embodiments or aspects shown in one drawing figure can be combined with one or more embodiments or aspects shown in one or more of the other drawing figures.

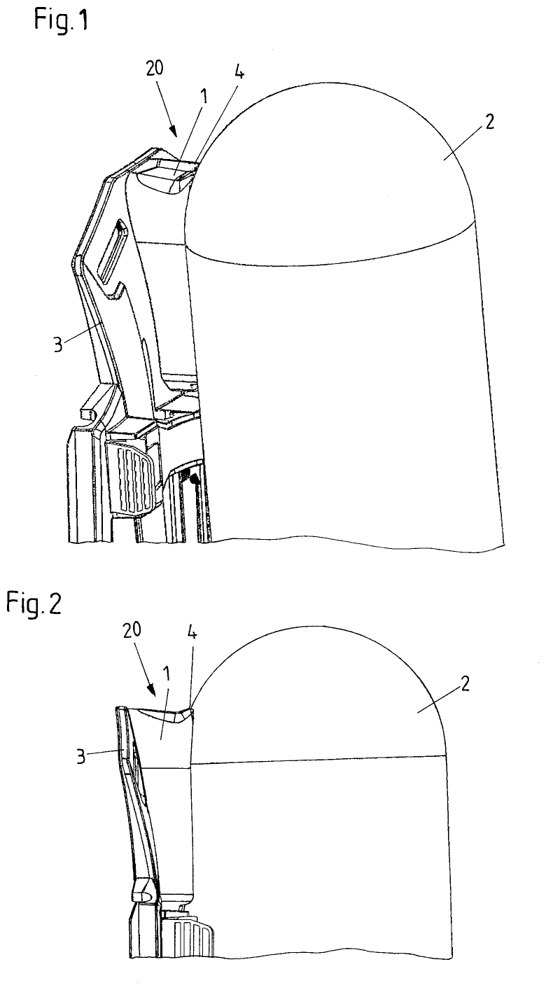

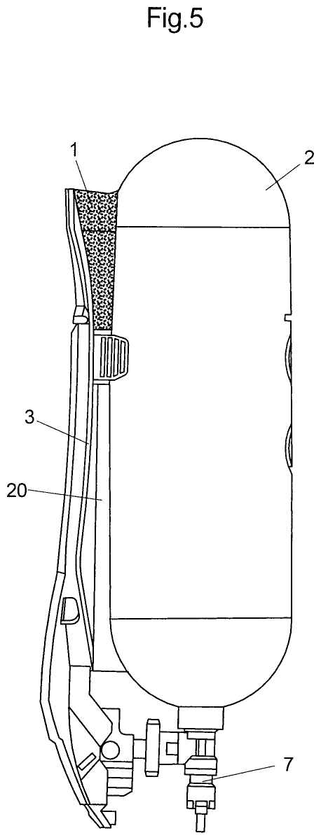

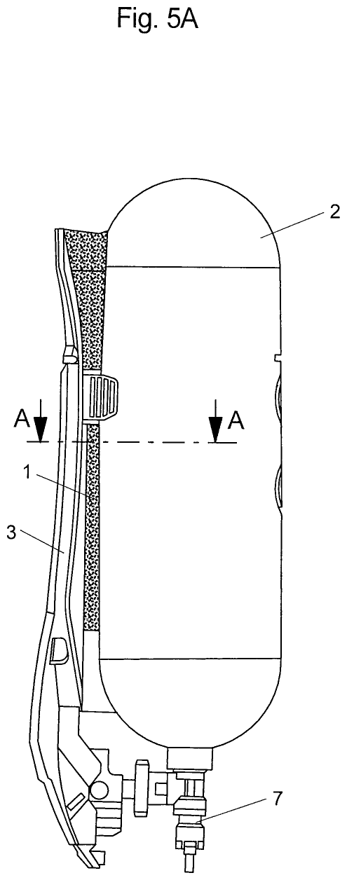

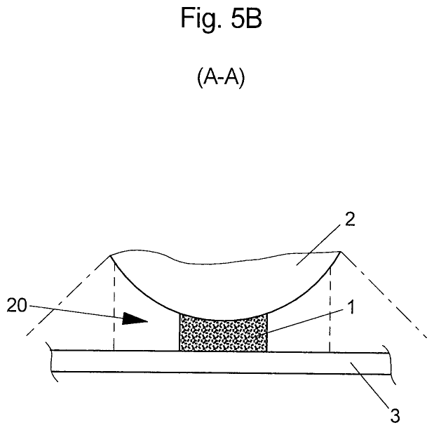

FIGS. 1 and 2 each show the upper part of a breathing apparatus (see also FIG. 5) from different perspectives in accordance with some non-limiting embodiments or aspects of the present disclosure. As shown, a compressed gas bottle 2 is connected with a carrier plate 3. The connection between compressed gas bottle 2 and carrier plate 3 typically is produced by means of straps or clamping devices, wherein these details have been omitted here for reasons of clarity. The carrier plate 3 is worn with corresponding shoulder straps by a person (not shown). FIGS. 1 and 2 each show the upper part of the breathing apparatus, i.e. the valve and the breathing gas ports at the lower end of the compressed gas bottle 2 are not visible here, but can be seen in FIGS. 5 and 5A.

In some non-limiting embodiments or aspects, between the compressed gas bottle 2 and the carrier plate 3 there is a space 20 that on one side is delimited by the carrier plate 20. The substantially cylindrical compressed gas bottle 2 delimits the space 20 to the rear (as seen from the wearing person). Towards the sides, the space 20 is open. However, for the purposes of this description, the space 20 can be understood such that in any case everything in a perpendicular projection of the compressed gas bottle 2 onto the carrier plate 3 belongs to the space 20. Thus, this space 20 extends downwards along the carrier plate 3 and the compressed gas bottle 2. The space 20 is U-shaped in cross-section. This is illustrated by broken lines, for example, in FIG. 5B. The space 20 also can extend laterally beyond the compressed gas bottle 2, up to the sides of the carrier plate 3, which is indicated in FIG. 5B by dash-dotted lines.

When the outer contour of the compressed gas bottle 2 deviates from the compressed gas bottle 2 shown here, the shape of the space 20 would change as well. The space 20 thus substantially represents a negative impression of the compressed gas bottle 2. In service, a cable or another item can get caught in this space 20, e.g. when creeping or when moving in a stooped posture, which can represent a dangerous situation.

FIGS. 1 and 2 therefore show a filling element 1 in accordance with some non-limiting embodiments or aspects, which is arranged in the upper region of the space 20 between compressed gas bottle 2 and carrier plate 3. This filling element 1 prevents items from getting into the space 20. The person wearing the breathing apparatus would notice very early that he or she hits an obstacle with the equipment and might change his or her position correspondingly. In some non-limiting embodiments or aspects, the filling element 1 is made of an elastomer so that the elastic deformability is specifically adjustable. In some non-limiting embodiments or aspects, another plastic material or a filling element 1 made of metal can be used. In some non-limiting embodiments or aspects, the filling element 1 is made of a composite material.

In particular, in FIG. 2 it can be seen that the filling element 1 terminates flush with the carrier plate 3 at the upper edge in some non-limiting embodiments or aspects. The upper side of the filling element 1 is of saddle-shaped design.

On the side towards the compressed gas bottle, the filling element 1 includes a first positive connection element 4 for connection with the compressed gas bottle 2. The filling element 1 as an elastomer conforms to the compressed gas bottle 2 so that a gap-free positive connection is obtained. When used as intended, the compressed gas bottle 2 is pressed against the carrier plate 3 by the non-illustrated straps so that this close fit is produced. The filling element 1 thus occupies a large part of the space 20 between the compressed gas bottle 2 and the carrier plate 3 so that no external item can penetrate into the space 20.

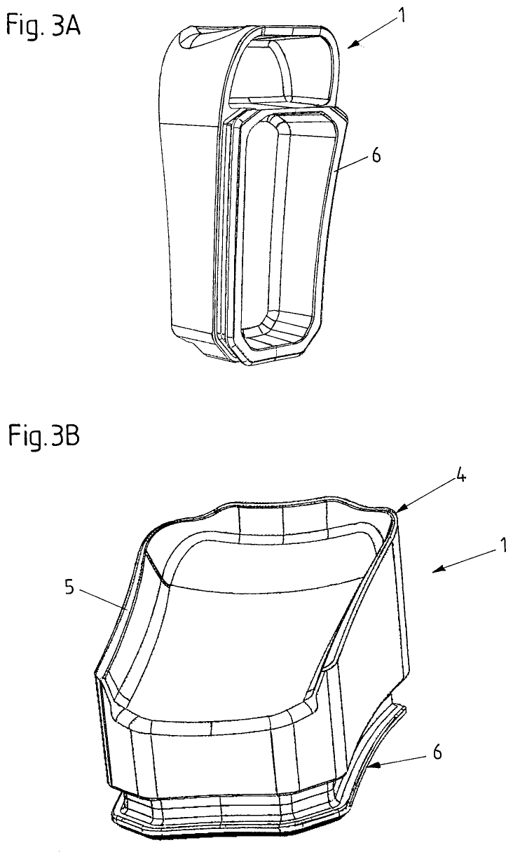

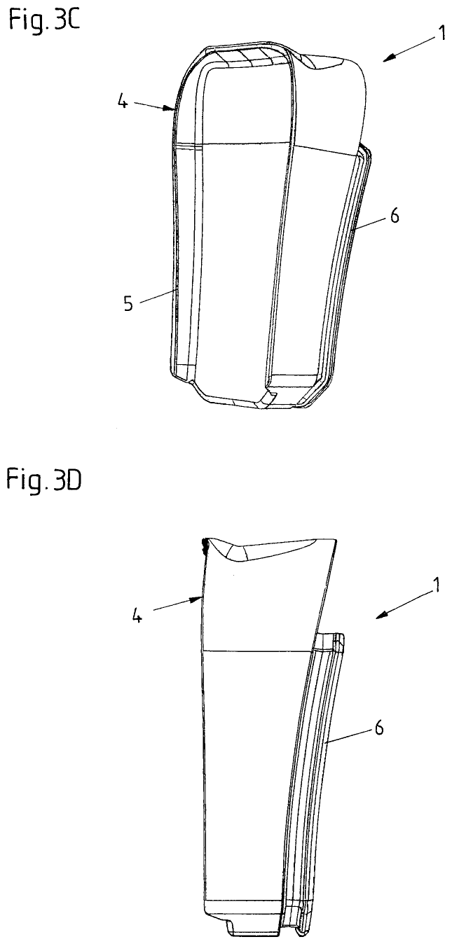

FIGS. 3A to 3D show some non-limiting embodiments or aspects of the filling element 1 of FIGS. 1 and 2 from different perspectives. FIG. 3A shows the rear view of the filling element 1, which, in assembly, faces the carrier plate 3. There, a second connecting element 6 is arranged, which is positively connectable with the carrier plate 3. The second positive connection element 6 is annularly arranged on the back, and thus forms a circumferential latching protrusion, which can make a latching, positive connection with a corresponding device of the carrier plate 3. In principle, other non-limiting embodiments or aspects also are conceivable. FIG. 3B shows the front side of the filling element 1, i.e. the side which, in assembly, faces the compressed gas bottle 2. In this view, the substantially trough-shaped, or concave construction of the front side, can be seen. At the top and on both sides, the trough is surrounded by the sealing lip 5, which allows the filling element 1 to conform to the compressed gas bottle. On the back of the filling element 1, the second connecting element 6 can be seen on the underside in this representation.

In the view of FIG. 3C, the trough-shaped construction of the filling element likewise is clearly visible. In the side view of FIG. 3D, the front side is shown on the left and the back side on the right. The filling element 1 according to the non-limiting embodiments or aspects in FIGS. 1 to 3 is formed substantially wedge-shaped in the outer contour, wherein the filling element 1 has a larger depth on the upper side than on the underside. This is particularly visible in FIGS. 2 and 3D.

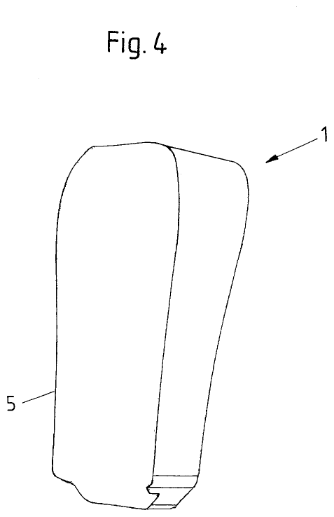

In some non-limiting embodiments or aspects, the filling element 1 has a broader design so that it conforms to a larger part of the circumference of the compressed gas bottle 2. This would provide a deeper trough-shaped configuration. In some non-limiting embodiments or aspects, the first positive connection means 4 has a sealing lip 5 which partly is arranged on the circumferential edge of the filling element 1 (see FIGS. 3A to 3D). In some non-limiting embodiments or aspects, the sealing lip 5 can be omitted, as the filling element 1 is constructed as a compact, substantially wedge-shaped body, as shown in FIG. 4. Here, the concave surface, which conforms to the compressed gas bottle 2, forms the positive connection element 4.

FIGS. 1 to 3 show some non-limiting embodiments or aspects of the filling element 1 that is arranged in the upper region of the carrier plate. In the longitudinal extension of the carrier plate 3, this filling element 1 extends downwards along less than about 20% of the length of the carrier plate. It is also possible, however, that the filling element 1 extends along more than a quarter of the extension of the carrier plate 3, in particular along half of the longitudinal extension of the carrier plate 3, particularly up to a port 7 of the at least one compressed gas bottle 2 at the lower end of the carrier plate 3. Such an arrangement is shown in FIG. 5A. The filling element 1 extends laterally in the space 20, almost up to the port 7. The filling element 1, thereby, prevents an external item from penetrating from the side.

FIG. 5B shows a section through the compressed gas bottle 2, the filling element 1, and the carrier plate 3. It can be seen that the space 20 between compressed gas bottle 2 and carrier plate 3 is partly filled up by the filling element 1. The lateral boundary of the space 20 is shown here by broken lines. The filling element 1, however, has even more possibilities of use, which, e.g., result from the fact that it is arranged at a point which is favorable in terms of wearing physiology, namely, close to the body of the person wearing the breathing apparatus. In FIG. 3A, for example, it can be seen that a filling element 1 has cavities in which functional units can be arranged. Due to the position between compressed gas bottle 2 and carrier plate 3, such functional units are arranged closed to the body of the person so as to be well-protected from external influences.

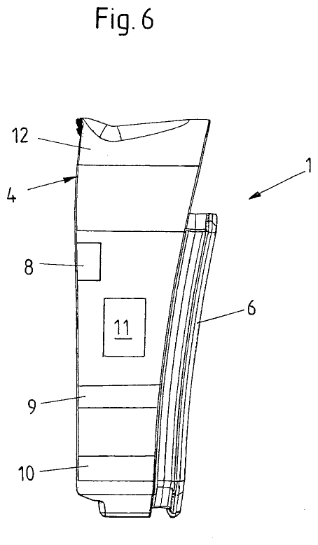

FIG. 6 shows a modification of the non-limiting embodiments or aspects of FIGS. 3A-D in which a number of functional units are accommodated. In some non-limiting embodiments or aspects, in the filling element 1, at least one gas sensor 8, in particular for toxic gases, can be arranged. As the filling element 1 can also be arranged, for example, in the vicinity of the head of the wearing person, a gas sensor 8 arranged there can measure relevant information, which can then trigger e.g. an acoustic signal. Measured values can also be transmitted to a display in a helmet (not shown here) via a wireless connection. In some non-limiting embodiments or aspects, the filling element 1 includes an indicating device 9 for an operating condition, for example a battery indicator or a level indicator, for the at least one compressed gas bottle 2. A lighting device, e.g., comprising LEDs, can also be provided on the filling element 1.

In the some non-limiting embodiments or aspects, the filling element 1 further includes at least one acoustic warning element 10, such as a loudspeaker, which is arranged well-protected at this point. In particular, in the case of a larger filling element 1, it is expedient to arrange a battery device 11 in the interior of the filling element 1. Furthermore, in some non-limiting embodiments or aspects, the filling element 1 includes a radio device 12, e.g., a locating means, a router, a means for producing an ad-hoc network, and/or the like. It is emphasized that FIG. 6 is to be understood merely by way of example, as not all functional units must necessarily be installed in one embodiment.

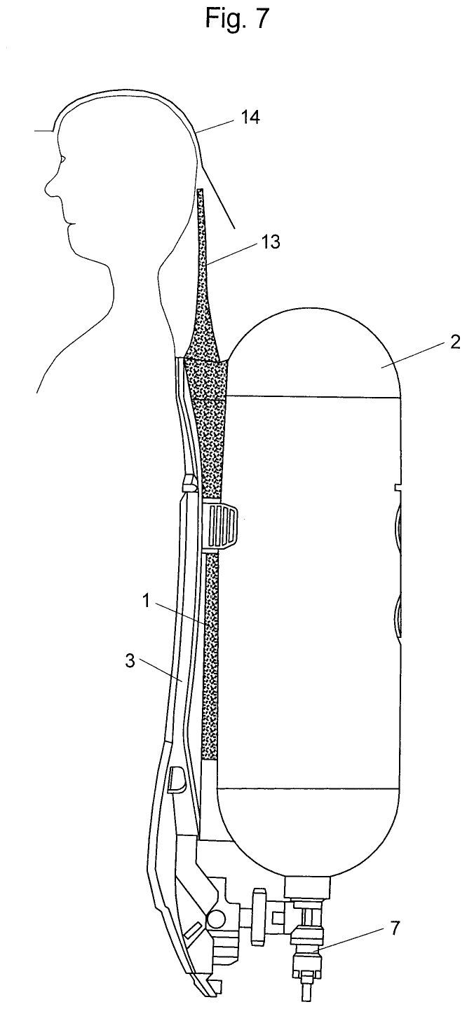

FIG. 7 shows some non-limiting embodiments or aspects analogous to the one of FIG. 5, wherein a neck guard 13 is integrated for a wearer of the breathing apparatus with the filling element 1. The neck guard 13 can cooperate with a helmet 14 of a wearer of the breathing apparatus in order to protect the sensitive neck area of a wearer. The neck guard 13 can be connected with the helmet 14, or overlap with a part of the helmet 14. FIG. 7 also shows that the filling element 1 may include a receiving element 8 for a hose line (not shown here). In some non-limiting embodiments or aspects, the receiving element 8 is configured as a depression into which a hose can be clamped.

For reasons of simplicity, the illustrated non-limiting embodiments or aspects of the filling element 1 are shown in connection with a breathing apparatus having only one compressed gas bottle 2. In some non-limiting embodiments or aspects, the filling element 1 can also be used with two or more compressed gas bottles 2 in order to fill up the respective space 20 towards the carrier plate 3. It should also be noted that, in some non-limiting embodiments or aspects, the filling element 1 at least partly can also protrude laterally beyond the projection of the compressed gas bottle 2 onto the carrier plate so that it is not only arranged in the space 20.

The non-limiting embodiments or aspects of the present disclosure have been described in detail herein. However, it will be appreciated by those skilled in the art that various modifications and alternatives to the embodiments or aspects may be made without departing from the concepts disclosed in the foregoing description. Such modifications are to be considered as included within the following claims unless the claims, by their language, expressly state otherwise. Accordingly, the particular embodiments or aspects described in detail hereinabove are illustrative only and are not limiting as to the scope of the disclosure, which is to be given the full breadth of the appended claims and any and all equivalents thereof.

1. A carrier for carrying at least one compressed gas bottle of a breathing apparatus, the carrier comprising:
a carrier plate configured for removably receiving the at least one compressed gas bottle; and
at least one filling element positioned in a space between the carrier plate and the at least one compressed air bottle,
wherein the at least one filling element comprises:
a first positive connection element having a surface for engaging the at least one compressed gas bottle, and
a second positive connection element connected to the first positive connection element and the carrier plate.

2. The carrier of claim 1, wherein at least one filling element has a length extending in a longitudinal direction along at least a quarter of a length of the carrier plate.

3. The carrier of claim 1, wherein the at least one filling element has a wedge shape with a larger depth on an upper side than a lower side.

4. The carrier of claim 1, wherein the surface of the first positive connection element is at least partially concave.

5. The carrier of claim 1, wherein the first positive connection element has a sealing lip configured for conforming to an outer surface of the at least one compressed gas bottle.

6. The carrier of claim 1, wherein the first positive connection element is made from an elastomer material that conforms to the at least one compressed gas bottle.

7. The carrier of claim 1, wherein the second positive connection element is latched into a groove of the carrier plate.

8. The carrier of claim 1, wherein the at least one filling element further comprises at least one functional unit.

9. The carrier of claim 8, wherein the at least one functional unit is received within a cavity of the at least one filling element.

10. The carrier of claim 8, wherein the at least one functional unit is at least one of: a gas sensor, an indicating device configured for indicating an operating condition, a lighting device, an acoustic warning element, a battery device, a radio device, and any combination thereof.

11. The carrier of claim 10, wherein the indicating device is at least one of a battery indicator and a level indicator for the at least one compressed gas bottle.

12. The carrier of claim 10, wherein the lighting device is configured for signaling a state of the breathing apparatus or as an emergency lighting.

13. The carrier of claim 1, wherein the at least one filling element further comprises at least one receiving element configured for receiving a breathing hose.

14. The carrier of claim 1, wherein the at least one receiving element is a depression.

15. The carrier of claim 1, wherein the at least one filling element further comprises a neck guard configured for cooperating with a helmet.

16. A breathing apparatus comprising:
at least one compressed gas bottle;
a carrier plate configured for removably receiving the at least one compressed gas bottle; and
at least one filling element positioned in a space between the carrier plate and the at least one compressed air bottle,
wherein the at least one filling element comprises:
a first positive connection element having a surface for engaging the at least one compressed gas bottle, and
a second positive connection element connected to the first positive connection element and the carrier plate.

17. The breathing apparatus of claim 16, wherein at least one filling element has a length extending in a longitudinal direction along at least a quarter of a length of the carrier plate, and wherein the at least one filling element has a wedge shape with a larger depth on an upper side than a lower side.

18. The breathing apparatus of claim 16, wherein the surface of the first positive connection element is at least partially concave and has a sealing lip configured for conforming to an outer surface of the at least one compressed gas bottle.

19. The breathing apparatus of claim 16, wherein the at least one filling element further comprises at least one functional unit received within a cavity of the at least one filling element, and wherein the at least one functional unit is at least one of: a gas sensor, an indicating device configured for indicating an operating condition, a lighting device, an acoustic warning element, a battery device, a radio device, and any combination thereof.

20. The breathing apparatus of claim 16, wherein the at least one filling element further comprises at least one receiving element configured for receiving a breathing hose.

21-54. (canceled)
